(19) United States
(12) Patent Application Publication
(10) Pub. No.: US 2025/0161555 A1
(43) Pub. Date: May 22, 2025

(54) APPARATUSES, KITS, AND METHODS FOR TISSUE INTERFACE PLACEMENT

(71) Applicant: Solventum Intellectual Properties Company, Maplewood, MN (US)

(72) Inventors: Javier GONZALEZ, Saint Paul, MN (US); Jonathan G. REHBEIN, Saint Paul, MN (US); Christopher J. CARROLL, Saint Paul, MN (US); Richard M. KAZALA, JR., Saint Paul, MN (US)

(21) Appl. No.: 18/841,223

(22) PCT Filed: Feb. 24, 2023
(86) PCT No.: PCT/IB2023/051738

§ 371 Date: Aug. 23, 2024

Related U.S. Application Data
(60) Provisional application No. 63/315,419, filed on Mar. 1, 2022.

(51) Int. Cl. A61M 1/00

(52) U.S. Cl. CPC A61M 1/966; A61M 1/918; A61M 2205/3368; A61M 2205/3584; A61M 2205/60

(57) ABSTRACT

Tissue interfaces, kits, and methods for treating an abdominal cavity. A tissue interface for use in an abdominal cavity includes a first contact layer, a second contact layer, a spacer layer, and a plurality of location tags. The first contact layer comprises a first plurality of perforations. The second contact layer comprises a second plurality of perforations. The spacer layer is disposed between the first contact layer and the second contact layer. The plurality of location tags are configured to be located by an identifier tool and to be positioned in a plurality of treatment areas within the abdominal cavity.

### CROSS-REFERENCE TO RELATED APPLICATIONS

This application is a U.S. National Stage Entry of International Application No. PCT/IB2023/051738, filed on Feb. 24, 2023, which claims the benefit of priority to U.S. Provisional Application No. 63/315,419, filed on Mar. 1, 2022, each of which are incorporated herein by reference in their entirety.

### TECHNICAL FIELD

The invention set forth in the appended claims relates generally to tissue treatment systems and more particularly, but without limitation, to apparatuses, kits, and methods for tissue interface placement using near field communication technology.

### BACKGROUND

Clinical studies and practice have shown that reducing pressure in proximity to a tissue site can augment and accelerate growth of new tissue at the tissue site. The applications of this phenomenon are numerous, but it has proven particularly advantageous for treating wounds. Regardless of the etiology of a wound, whether trauma, surgery, or another cause, proper care of the wound is important to the outcome. Treatment of wounds or other tissue with reduced pressure may be commonly referred to as “negative-pressure therapy,” but is also known by other names, including “negative-pressure wound therapy,” “reduced-pressure therapy,” “vacuum therapy,” “vacuum-assisted closure,” and “topical negative-pressure,” for example. Negative-pressure therapy may provide a number of benefits, including migration of epithelial and subcutaneous tissues, improved blood flow, and micro-deformation of tissue at a wound site. Together, these benefits can increase development of granulation tissue and reduce healing times.

While the clinical benefits of negative-pressure therapy are widely known, improvements to therapy systems, components, and processes may benefit healthcare providers and patients.

### BRIEF SUMMARY

New and useful systems, apparatuses, and methods for placement of tissue interfaces using near field communication technology in a negative-pressure therapy environment are set forth in the appended claims. Illustrative embodiments are also provided to enable a person skilled in the art to make and use the claimed subject matter.

For example, in some embodiments, a tissue interface for use in an abdominal cavity is described. The tissue interface can include a first contact layer, a second contact layer, a spacer layer, and a plurality of location tags. The first contact layer can include a first plurality of perforations and the second contact layer can include a second plurality of perforations. The spacer layer can be disposed between the first contact layer and the second contact layer. The plurality of location tags can be configured to be located by an identifier tool and can be positioned in a plurality of treatment areas within the abdominal cavity.

In some example embodiments, the spacer layer can include a central portion and a plurality of appendages radiating from the central portion. The plurality of appendages can be configured to extend to a plurality of zones within the abdominal cavity. The plurality of location tags can be disposed between the plurality of appendages of the spacer layer.

In some example embodiments, each location tag of the plurality of location tags can be a near field communication (NFC) device. Each location tag of the plurality of location tags can be encoded with a unique identifier. In some embodiments, the plurality of location tags can be disposed between the first contact layer and the second contact layer such that the plurality of location tags are isolated from the abdominal cavity by the first contact layer and the second contact layer.

In some example embodiments, the identifier tool can be configured to locate each location tag of the plurality of location tags separately from another location tag of the plurality of location tags. The identifier tool can be configured to locate one of the location tags of the plurality of location tags when the identifier tool is within a sensing distance the one of the location tags.

In some embodiments, the location tags can be configured to collect treatment data associated with the plurality of treatment areas within the abdominal cavity. The treatment data can include at least one of a pressure in the abdominal cavity, a temperature in the abdominal cavity, a pH in the abdominal cavity, or a presence of fluid in the abdominal cavity. The plurality of location tags can be configured to communicate the treatment data with the identifier tool in some example embodiments.

A tissue interface kit is also described herein. The tissue interface kit can include a tissue interface and an identifier tool. The tissue interface can include a first contact layer, a second contact layer, a spacer layer, and a plurality of location tags. The first contact layer can include a first plurality of perforations and the second contact layer can include a second plurality of perforations. The spacer layer can be disposed between the first contact layer and the second contact layer. The plurality of location tags can be disposed between the first contact layer and the second contact layer and can be positioned in a plurality of treatment areas within the abdominal cavity. The identifier tool can be configured to sense a location of one of the plurality of location tags of the tissue interface at each of the treatment areas.

In some example embodiments the identifier tool can include a handheld device including a locator and an indicator configured to actuate when the locator is within a sensing distance of one of the location tags of the plurality of location tags. In another example embodiment the identifier tool can include a smart phone configured to communicate with each location tag of the plurality of location tags.

In some example embodiments, the location tags can be configured to collect treatment data from each of the plurality of treatment areas within the abdominal cavity. The identifier tool can be configured to display the treatment data of one of the location tags of the plurality of location tags when the identifier tool is within a sensing distance of the one of the location tags of the plurality of location tags.

In some example embodiments, each location tag of the plurality of location tags can be encoded with a unique identifier. The identifier tool can be configured to display the unique identifier of one of the location tags of the plurality of location tags when the identifier tool is within a sensing distance of the one of the location tags of the plurality of location tags.

A method of placing a tissue interface into an abdominal cavity is also described herein. The method can include inserting the tissue interface into the abdominal cavity. The tissue interface can include a first contact layer, a second contact layer, a spacer layer, and a plurality of location tags. The first contact layer can include a first plurality of perforations and the second contact layer can include a second plurality of perforations. The spacer layer and the plurality of location tags can be disposed between the first contact layer and the second contact layer. The method can further include locating, with an identifier tool, each location tag of the plurality of location tags to determine an orientation of the tissue interface in the abdominal cavity and adjusting the orientation of the tissue interface to place the tissue interface in a plurality of treatment areas within the abdominal cavity.

In some example embodiments, the method can further include collecting treatment data associated with the abdominal cavity from each location tag of the plurality of location tags using the identifier tool. The method can further include locating a unique identifier of each of the plurality of location tags at each of the plurality of treatment areas using the identifier tool. The method can further include assigning the treatment data to each of the plurality of treatment areas using the unique identifier and outputting, with the identifier tool, the treatment data at each of the plurality of treatment areas.

Objectives, advantages, and a preferred mode of making and using the claimed subject matter may be understood best by reference to the accompanying drawings in conjunction with the following detailed description of illustrative embodiments.

### BRIEF DESCRIPTION OF THE DRAWINGS

FIG. 1 is a block diagram of an example embodiment of a therapy system that can provide negative-pressure treatment in accordance with this specification;

FIG. 2 is an exploded view of an example tissue interface of the therapy system of FIG. 1;

FIG. 3 is a top view of the tissue interface of FIG. 2;

FIG. 4 is a perspective view of an identifier tool interacting with the tissue interface of FIG. 2;

FIG. 5 is a perspective view of another embodiment of the identifier tool interacting with the tissue interface of FIG. 2; and

FIG. 6 is a cut away view of the therapy system of FIG. 1 deployed at a tissue site.

### DESCRIPTION OF EXAMPLE EMBODIMENTS

The following description of example embodiments provides information that enables a person skilled in the art to make and use the subject matter set forth in the appended claims, but it may omit certain details already well-known in the art. The following detailed description is, therefore, to be taken as illustrative and not limiting.

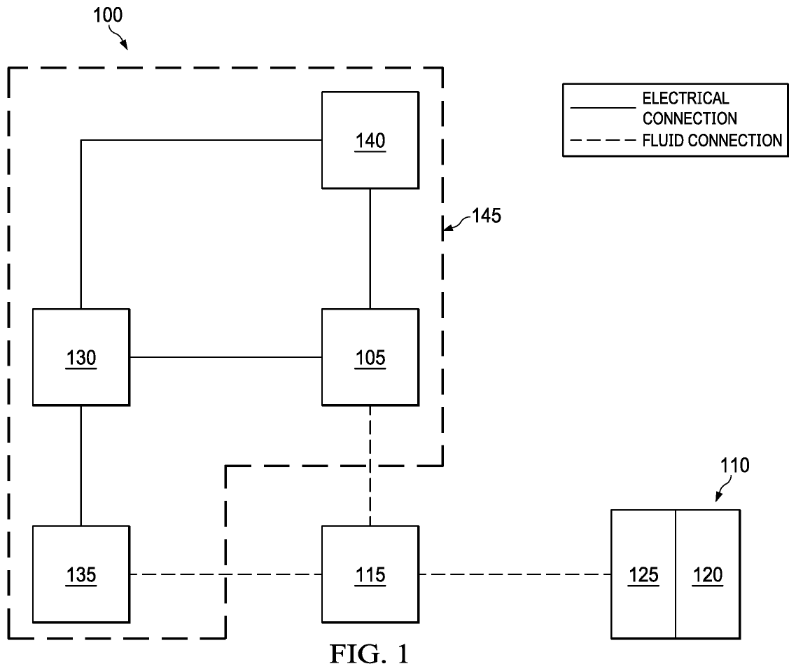

FIG. 1 is a block diagram of an example embodiment of a therapy system 100 that can provide negative-pressure therapy to a tissue site in accordance with this specification.

The term “tissue site” in this context broadly refers to a wound, defect, or other treatment target located on or within tissue, including, but not limited to, bone tissue, adipose tissue, muscle tissue, neural tissue, dermal tissue, vascular tissue, connective tissue, cartilage, tendons, or ligaments. A wound may include chronic, acute, traumatic, subacute, and dehisced wounds, partial-thickness burns, ulcers (such as diabetic, pressure, or venous insufficiency ulcers), flaps, and grafts, for example. The term “tissue site” may also refer to areas of any tissue that are not necessarily wounded or defective, but are instead areas in which it may be desirable to add or promote the growth of additional tissue. For example, negative pressure may be applied to a tissue site to grow additional tissue that may be harvested and transplanted.

Referring to FIG. 1, the therapy system 100 may include a source or supply of negative pressure, such as a negative-pressure source 105, and one or more distribution components. A distribution component is preferably detachable and may be disposable, reusable, or recyclable. A dressing, such as a dressing 110, and a fluid container, such as a container 115, are examples of distribution components that may be associated with some examples of the therapy system 100. As illustrated in the example of FIG. 1, the dressing 110 may comprise or consist essentially of a tissue interface 120, a cover 125, or both in some embodiments.

A fluid conductor is another illustrative example of a distribution component. A “fluid conductor,” in this context, broadly includes a tube, pipe, hose, conduit, or other structure with one or more lumina or open pathways adapted to convey a fluid between two ends. Typically, a tube is an elongated, cylindrical structure with some flexibility, but the geometry and rigidity may vary. Moreover, some fluid conductors may be molded into or otherwise integrally combined with other components. Distribution components may also include or comprise interfaces or fluid ports to facilitate coupling and de-coupling other components. In some embodiments, for example, a dressing interface may facilitate coupling a fluid conductor to the dressing 110. For example, such a dressing interface may be a SENSAT.R.A.C.™ Pad available from Kinetic Concepts, Inc. of San Antonio, Texas.

The therapy system 100 may also include a regulator or controller, such as a controller 130. Additionally, the therapy system 100 may include sensors to measure operating parameters and provide feedback signals to the controller 130 indicative of the operating parameters. As illustrated in FIG. 1, for example, the therapy system 100 may include a first sensor 135 and a second sensor 140 coupled to the controller 130.

Some components of the therapy system 100 may be housed within or used in conjunction with other components, such as sensors, processing units, alarm indicators, memory, databases, software, display devices, or user interfaces that further facilitate therapy. For example, in some embodiments, the negative-pressure source 105 may be combined with the controller 130 and other components into a therapy unit 145.

In general, components of the therapy system 100 may be coupled directly or indirectly. For example, the negative-pressure source 105 may be directly coupled to the container 115 and may be indirectly coupled to the dressing 110 through the container 115. Coupling may include fluid, mechanical, thermal, electrical, or chemical coupling (such as a chemical bond), or some combination of coupling in some contexts. For example, the negative-pressure source 105 may be electrically coupled to the controller 130 and may be fluidly coupled to one or more distribution components to provide a fluid path to a tissue site. In some embodiments, components may also be coupled by virtue of physical proximity, being integral to a single structure, or being formed from the same piece of material.

A negative-pressure supply, such as the negative-pressure source 105, may be a reservoir of air at a negative pressure or may be a manual or electrically-powered device, such as a vacuum pump, a suction pump, a wall suction port available at many healthcare facilities, or a micro-pump, for example. “Negative pressure” generally refers to a pressure less than a local ambient pressure, such as the ambient pressure in a local environment external to a sealed therapeutic environment. In many cases, the local ambient pressure may also be the atmospheric pressure at which a tissue site is located. Alternatively, the pressure may be less than a hydrostatic pressure associated with tissue at the tissue site. Unless otherwise indicated, values of pressure stated herein are gauge pressures. References to increases in negative pressure typically refer to a decrease in absolute pressure, while decreases in negative pressure typically refer to an increase in absolute pressure. While the amount and nature of negative pressure provided by the negative-pressure source 105 may vary according to therapeutic requirements, the pressure is generally a low vacuum, also commonly referred to as a rough vacuum, between −5 mm Hg (−667 Pa) and −500 mm Hg (−66.7 kPa). Common therapeutic ranges are between −50 mm Hg (−6.7 kPa) and −300 mm Hg (−39.9 kPa).

The container 115 is representative of a container, canister, pouch, or other storage component, which can be used to manage exudates and other fluids withdrawn from a tissue site. In many environments, a rigid container may be preferred or required for collecting, storing, and disposing of fluids. In other environments, fluids may be properly disposed of without rigid container storage, and a re-usable container could reduce waste and costs associated with negative-pressure therapy.

A controller, such as the controller 130, may be a microprocessor or computer programmed to operate one or more components of the therapy system 100, such as the negative-pressure source 105. In some embodiments, for example, the controller 130 may be a microcontroller, which generally comprises an integrated circuit containing a processor core and a memory programmed to directly or indirectly control one or more operating parameters of the therapy system 100. Operating parameters may include the power applied to the negative-pressure source 105, the pressure generated by the negative-pressure source 105, or the pressure distributed to the tissue interface 120, for example. The controller 130 is also preferably configured to receive one or more input signals, such as a feedback signal, and programmed to modify one or more operating parameters based on the input signals.

Sensors, such as the first sensor 135 and the second sensor 140, are generally known in the art as any apparatus operable to detect or measure a physical phenomenon or property, and generally provide a signal indicative of the phenomenon or property that is detected or measured. For example, the first sensor 135 and the second sensor 140 may be configured to measure one or more operating parameters of the therapy system 100. In some embodiments, the first sensor 135 may be a transducer configured to measure pressure in a pneumatic pathway and convert the measurement to a signal indicative of the pressure measured. In some embodiments, for example, the first sensor 135 may be a piezo-resistive strain gauge. The second sensor 140 may optionally measure operating parameters of the negative-pressure source 105, such as a voltage or current, in some embodiments. The signals from the first sensor 135 and the second sensor 140 may be suitable as an input signal to the controller 130, but some signal conditioning may be appropriate in some embodiments. For example, the signal may need to be filtered or amplified before it can be processed by the controller 130. Typically, the signal is an electrical signal, but may be represented in other forms, such as an optical signal.

The tissue interface 120 can be generally adapted to partially or fully contact a tissue site. The tissue interface 120 may take many forms, and may have many sizes, shapes, or thicknesses, depending on a variety of factors, such as the type of treatment being implemented or the nature and size of a tissue site. For example, the size and shape of the tissue interface 120 may be adapted to the contours of deep and irregular shaped tissue sites. Any or all of the surfaces of the tissue interface 120 may have an uneven, coarse, or jagged profile.

In some embodiments, the tissue interface 120 may comprise or consist essentially of a manifold. A manifold in this context may comprise or consist essentially of a means for collecting or distributing fluid across the tissue interface 120 under pressure. For example, a manifold may be adapted to receive negative pressure from a source and distribute negative pressure through multiple apertures across the tissue interface 120, which may have the effect of collecting fluid from across a tissue site and drawing the fluid toward the source. In some embodiments, the fluid path may be reversed, or a secondary fluid path may be provided to facilitate delivering fluid across a tissue site.

In some illustrative embodiments, a manifold may comprise a plurality of pathways, which can be interconnected to improve distribution or collection of fluids. In some illustrative embodiments, a manifold may comprise or consist essentially of a porous material having interconnected fluid pathways. Examples of suitable porous material that can be adapted to form interconnected fluid pathways (e.g., channels) may include cellular foam, including open-cell foam such as reticulated foam; porous tissue collections; and other porous material such as gauze or felted mat that generally include pores, edges, and/or walls. Liquids, gels, and other foams may also include or be cured to include apertures and fluid pathways. In some embodiments, a manifold may additionally or alternatively comprise projections that form interconnected fluid pathways. For example, a manifold may be molded to provide surface projections that define interconnected fluid pathways.

In some embodiments, the tissue interface 120 may comprise or consist essentially of reticulated foam having pore sizes and free volume that may vary according to needs of a prescribed therapy. For example, reticulated foam having a free volume of at least 90% may be suitable for many therapy applications, and foam having an average pore size in a range of 400-600 microns (40-50 pores per inch) may be particularly suitable for some types of therapy. The tensile strength of the tissue interface 120 may also vary according to needs of a prescribed therapy. For example, the tensile strength of foam may be increased for instillation of topical treatment solutions. The 25% compression load deflection of the tissue interface 120 may be at least 0.35 pounds per square inch, and the 65% compression load deflection may be at least 0.43 pounds per square inch. In some embodiments, the tensile strength of the tissue interface 120 may be at least 10 pounds per square inch. The tissue interface 120 may have a tear strength of at least 2.5 pounds per inch. In some embodiments, the tissue interface may be foam comprised of polyols such as polyester or polyether, isocyanate such as toluene diisocyanate, and polymerization modifiers such as amines and tin compounds. In some examples, the tissue interface 120 may be reticulated polyurethane foam such as found in GRANUFOAM™ dressing or V.A.C. VERAFLO™ dressing, both available from Kinetic Concepts, Inc. of San Antonio, Texas.

The thickness of the tissue interface 120 may also vary according to needs of a prescribed therapy. For example, the thickness of the tissue interface may be decreased to reduce tension on peripheral tissue. The thickness of the tissue interface 120 can also affect the conformability of the tissue interface 120. In some embodiments, a thickness in a range of about 5 millimeters to 10 millimeters may be suitable.

The tissue interface 120 may be either hydrophobic or hydrophilic. In an example in which the tissue interface 120 may be hydrophilic, the tissue interface 120 may also wick fluid away from a tissue site, while continuing to distribute negative pressure to the tissue site. The wicking properties of the tissue interface 120 may draw fluid away from a tissue site by capillary flow or other wicking mechanisms. An example of a hydrophilic material that may be suitable is a polyvinyl alcohol, open-cell foam such as V.A.C. WHITEFOAM™ dressing available from Kinetic Concepts, Inc. of San Antonio, Texas. Other hydrophilic foams may include those made from polyether. Other foams that may exhibit hydrophilic characteristics include hydrophobic foams that have been treated or coated to provide hydrophilicity.

In some embodiments, the tissue interface 120 may be constructed from bioresorbable materials. Suitable bioresorbable materials may include, without limitation, a polymeric blend of polylactic acid (PLA) and polyglycolic acid (PGA). The polymeric blend may also include, without limitation, polycarbonates, polyfumarates, and capralactones. The tissue interface 120 may further serve as a scaffold for new cell-growth, or a scaffold material may be used in conjunction with the tissue interface 120 to promote cell-growth. A scaffold is generally a substance or structure used to enhance or promote the growth of cells or formation of tissue, such as a three-dimensional porous structure that provides a template for cell growth. Illustrative examples of scaffold materials include calcium phosphate, collagen, PLA/PGA, coral hydroxy apatites, carbonates, or processed allograft materials.

In some embodiments, the cover 125 may provide a bacterial barrier and protection from physical trauma. The cover 125 may also be constructed from a material that can reduce evaporative losses and provide a fluid seal between two components or two environments, such as between a therapeutic environment and a local external environment. The cover 125 may comprise or consist of, for example, an elastomeric film or membrane that can provide a seal adequate to maintain a negative pressure at a tissue site for a given negative-pressure source. The cover 125 may have a high moisture-vapor transmission rate (MVTR) in some applications. For example, the MVTR may be at least 250 grams per square meter per twenty-four hours in some embodiments, measured using an upright cup technique according to ASTM E96/E96M Upright Cup Method at 38° C. and 10% relative humidity (RH). In some embodiments, an MVTR up to 5,000 grams per square meter per twenty-four hours may provide effective breathability and mechanical properties.

In some example embodiments, the cover 125 may be a polymer drape, such as a polyurethane film, that is permeable to water vapor but impermeable to liquid. Such drapes typically have a thickness in the range of 25-50 microns. For permeable materials, the permeability generally should be low enough that a desired negative pressure may be maintained. The cover 125 may comprise, for example, one or more of the following materials: polyurethane (PU), such as hydrophilic polyurethane; cellulosics; hydrophilic polyamides; polyvinyl alcohol; polyvinyl pyrrolidone; hydrophilic acrylics; silicones, such as hydrophilic silicone elastomers; natural rubbers; polyisoprene; styrene butadiene rubber; chloroprene rubber; polybutadiene; nitrile rubber; butyl rubber; ethylene propylene rubber; ethylene propylene diene monomer; chlorosulfonated polyethylene; polysulfide rubber; ethylene vinyl acetate (EVA); co-polyester; and polyether block polymide copolymers. Such materials are commercially available as, for example, Tegaderm® drape, commercially available from 3M Company, Minneapolis Minnesota; polyurethane (PU) drape, commercially available from Avery Dennison Corporation, Pasadena, California; polyether block polyamide copolymer (PEBAX), for example, from Arkema S.A., Colombes, France; and Inspire 2301 and Inpsire 2327 polyurethane films, commercially available from Expopack Advanced Coatings, Wrexham, United Kingdom. In some embodiments, the cover 125 may comprise INSPIRE 2301 having an MVTR (upright cup technique) of 2600 g/m²/24 hours and a thickness of about 30 microns.

An attachment device may be used to attach the cover 125 to an attachment surface, such as undamaged epidermis, a gasket, or another cover. The attachment device may take many forms. For example, an attachment device may be a medically-acceptable, pressure-sensitive adhesive configured to bond the cover 125 to epidermis around a tissue site. In some embodiments, for example, some or all of the cover 125 may be coated with an adhesive, such as an acrylic adhesive, which may have a coating weight of about 25-65 grams per square meter (g.s.m.). Thicker adhesives, or combinations of adhesives, may be applied in some embodiments to improve the seal and reduce leaks. Other example embodiments of an attachment device may include a double-sided tape, paste, hydrocolloid, hydrogel, silicone gel, or organogel.

In operation, the tissue interface 120 may be placed within, over, on, or otherwise proximate to a tissue site. If the tissue site is a wound, for example, the tissue interface 120 may partially or completely fill the wound, or it may be placed over the wound. The cover 125 may be placed over the tissue interface 120 and sealed to an attachment surface near a tissue site. For example, the cover 125 may be sealed to undamaged epidermis peripheral to a tissue site. Thus, the dressing 110 can provide a sealed therapeutic environment proximate to a tissue site, substantially isolated from the external environment, and the negative-pressure source 105 can reduce pressure in the sealed therapeutic environment.

The process of reducing pressure may be described illustratively herein as “delivering,” “distributing,” or “generating” negative pressure, for example. In general, exudate and other fluid flow toward lower pressure along a fluid path. Thus, the term “downstream” typically implies a location in a fluid path relatively closer to a source of negative pressure or further away from a source of positive pressure. Conversely, the term “upstream” implies a location relatively further away from a source of negative pressure or closer to a source of positive pressure. However, the fluid path may also be reversed in some applications, such as by substituting a positive-pressure source for a negative-pressure source.

Negative pressure applied to the tissue site through the tissue interface 120 in the sealed therapeutic environment can induce macro-strain and micro-strain in the tissue site. Negative pressure can also remove exudate and other fluid from a tissue site, which can be collected in the container 115.

In some embodiments, the controller 130 may receive and process data from one or more sensors, such as the first sensor 135. The controller 130 may also control the operation of one or more components of the therapy system 100 to manage the pressure delivered to the tissue interface 120. In some embodiments, the controller 130 may include an input for receiving a desired target pressure and may be programmed for processing data relating to the setting and inputting of the target pressure to be applied to the tissue interface 120. In some example embodiments, the target pressure may be a fixed pressure value set by an operator as the target negative pressure desired for therapy at a tissue site and then provided as input to the controller 130. The target pressure may vary from tissue site to tissue site based on the type of tissue forming a tissue site, the type of injury or wound (if any), the medical condition of the patient, and the preference of the attending physician. After selecting a desired target pressure, the controller 130 can operate the negative-pressure source 105 in one or more control modes based on the target pressure and may receive feedback from one or more sensors to maintain the target pressure at the tissue interface 120.

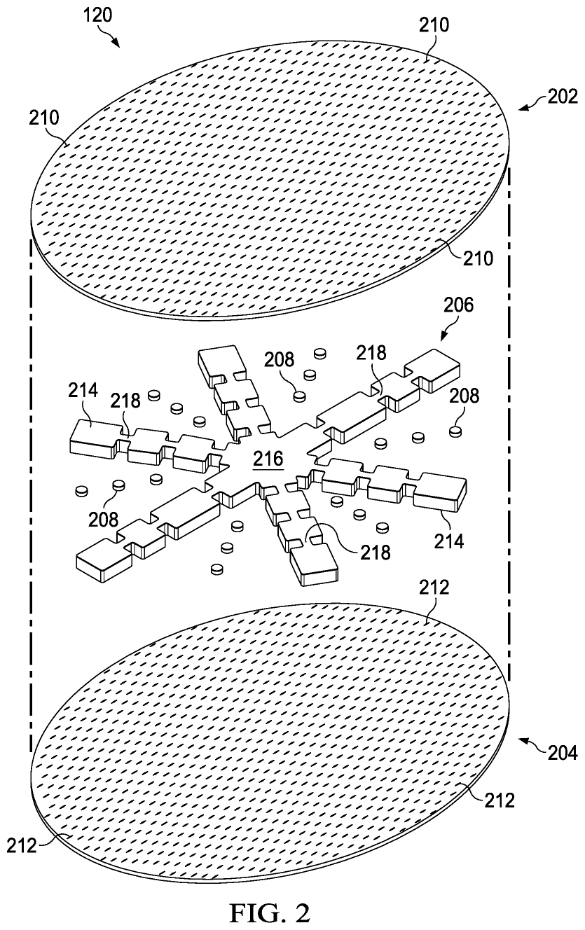

FIG. 2 depicts an exploded view of an example embodiment of the tissue interface 120 of the therapy system 100 for use in an abdominal cavity. The tissue interface 120 may include a first contact layer 202, a second contact layer 204, a spacer layer 206, and a plurality of sensors or a plurality of location tags 208. The spacer layer 206 may be disposed between the first contact layer 202 and the second contact layer 204. In some embodiments, the plurality of location tags 208 may be disposed between the first contact layer 202 and the second contact layer 204 such that each location tag 208 of the plurality of location tags 208 is isolated from the abdominal cavity by the first contact layer 202 and the second contact layer 204. In other embodiments, the plurality of location tags 208 may be disposed in a different location relative to the first contact layer 202 and the second contact layer 204.

Each of the first contact layer 202, the second contact layer 204, and the spacer layer 206 may be a manifold. For example, as illustrated in FIG. 2, the first contact layer 202 and the second contact layer 204 may have slits, fenestrations, or perforations suitable for distributing or collecting fluid across the tissue interface 120. For example, the first contact layer 202 may have a first plurality of perforations 210 and the second contact layer 204 may have a second plurality of perforations 212. The first plurality of perforations 210 and the second plurality of perforations 212 can have a variety of suitable shapes. For example, the first plurality of perforations 210 and the second plurality of perforations 212 may be circular or rectangular. In FIG. 2, the first plurality of perforations 210 and the second plurality of perforations 212 are slits.

The first contact layer 202 and the second contact layer 204 may be sufficiently flexible to conform to a tissue site. For example, the first contact layer 202 and the second contact layer 204 may be a thin film of construction similar to the cover 125. A thickness of about 50 microns to about 120 microns may be suitable for some embodiments of the first contact layer 202 and the second contact layer 204.

Similar to the first contact layer 202 and the second contact layer 204, the spacer layer 206 may be sufficiently flexible to conform to the abdominal cavity. In some examples, the spacer layer 206 may be formed from a porous material, such as open-cell foam. In other embodiments, the spacer layer 206 may be formed from another flexible foam. The profile of the spacer layer 206 may also provide flexibility. In the example of FIG. 2, the spacer layer 206 has a star profile having a plurality of appendages, such as spacer legs 214, coupled to and radiating from a central portion or a central body 216. When the tissue interface 120 is disposed within the abdominal cavity, the spacer legs 214 may be configured to extend to a plurality of zones within the abdominal cavity. The spacer legs 214 can be manipulated to conform to various types of tissues sites having complex geometries. Other suitable profiles of the spacer layer 206 may include interconnected concentric rings or arcs, or some combination of appendages, rings, and arcs, which may be coupled to or form the central body 216. In some examples, the spacer legs 214 or other appendages may comprise a plurality of joints 218, which can further increase flexibility.

The plurality of location tags 208 may be positioned between the spacer legs 214 of the spacer layer 206 and may be located in a plurality of treatment areas when the tissue interface 120 is disposed within a tissue site. For example, when the tissue interface 120 is disposed within the abdominal cavity, the plurality of location tags 208 may be positioned in a plurality of treatment areas within the abdominal cavity. In some embodiments, each location tag 208 of the plurality of location tags 208 may be a near field communication (NFC) device. NFC devices may be useful because they do not need a power source but rather leverage the power of an NFC reading device. Thus, when the plurality of location tags 208 are NFC devices, there are no additional power sources or additional electronics included in the tissue interface 120. Each location tag 208 of the plurality of location tags 208 may be encoded with a unique identifier, such as a code, number, symbol, or any other suitable identifier or combination of identifiers. The unique identifier may be identified when the plurality of location tags 208 are identified by an NFC reading device. The unique identifier of each location tag 208 of the plurality of location tags 208 may enable a user or a health care provider to determine the placement of the tissue interface 120 within the abdominal cavity.

In some embodiments, each location tag 208 of the plurality of location tags 208 may further be configured to collect treatment data associated with the plurality of treatment areas within the abdominal cavity. The treatment data may include at least one of a pressure in the abdominal cavity, a temperature in the abdominal cavity, a pH in the abdominal cavity, or a presence of fluid in the abdominal cavity. In other embodiments, the plurality of location tags 208 may be configured to collect additional information associated with the plurality of treatment areas within the abdominal cavity. Similar to the unique identifier of each location tag 208 of the plurality of location tags 208, the treatment data collected by each location tag 208 of the plurality of location tags 208 may be communicated to an NFC reading device when each location tag 208 of the plurality of location tags 208 is identified by an NFC reading device.

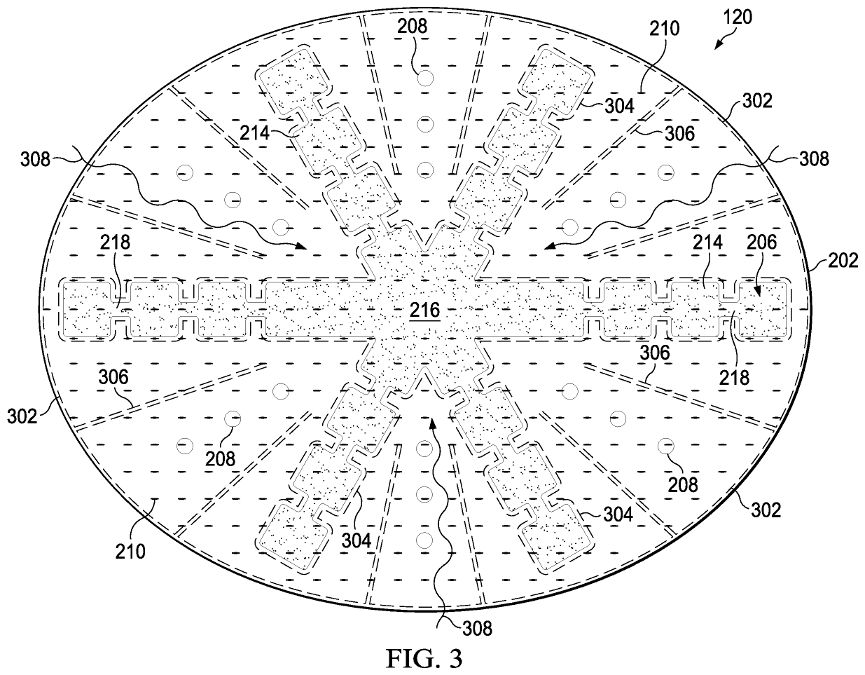

FIG. 3 is a top view of the tissue interface 120 of FIG. 2, as assembled, illustrating additional details that may be associated with some examples. The second contact layer 204 (not visible) may be geometrically similar to the first contact layer 202 and in some embodiments, the first contact layer 202 may be congruent to the second contact layer 204. A plurality of bonds may be used to couple the first contact layer 202 to the second contact layer 204. The bonds may be formed using known techniques, including without limitation, welding (e.g., ultrasonic or RF welding), bonding, adhesives, cements, or other bonding technique or apparatus. In the example of FIG. 3, the bonds include peripheral bonds 302, spacer bonds 304, and directional bonds 306.

The peripheral bonds 302 may be disposed around a periphery of the first contact layer 202 and the second contact layer 204. The spacer bonds 304 can be disposed around the spacer layer 206, which can secure the spacer layer 206 in a fixed position relative to the first contact layer 202 and the second contact layer 204. In some embodiments, the directional bonds 306 can define one or more flow paths 308 toward the central body 216. For example, the directional bonds 306 are disposed between the spacer legs 214, and generally extend radially between the central body 216 and the peripheral bonds 302.

The plurality of location tags 208 may be disposed within the flow paths 308 such that they are located between the spacer legs 214 of the spacer layer 206. In some embodiments, there may be additional bonds disposed around each location tag 208 to secure each location tag 208 in a fixed position relative to the first contact layer 202 and the second contact layer 204.

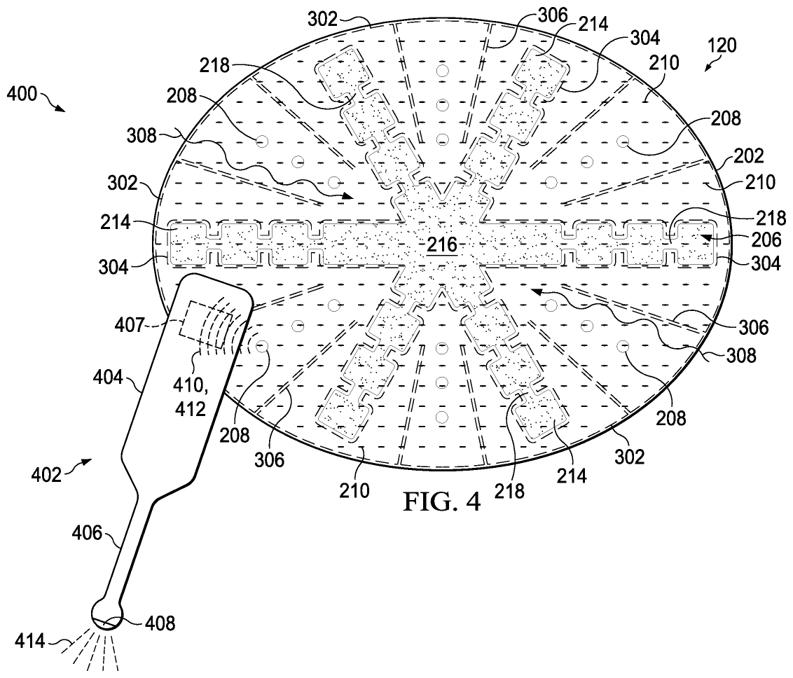

FIG. 4 is a perspective view of a tissue interface kit 400. The tissue interface kit 400 may include an NFC reading device or an identifier tool 402 and the tissue interface 120 of FIGS. 2 and 3. The identifier tool 402 may be a handheld device that may be configured to interact with the plurality of location tags 208 of the tissue interface 120. The identifier tool 402 may include a locator or a sensing portion 404 and a handle 406. The sensing portion 404 may be or may include an NFC reader 407 configured to interact with the plurality of location tags 208 of the tissue interface 120. In some embodiments, the NFC reader 407 of the identifier tool 402 may be configured to locate each location tag 208 of the plurality of location tags 208 separately from another location tag 208 of the plurality of location tags 208. The NFC reader 407 of the identifier tool 402 may be used to determine where the tissue interface 120 is located within an abdominal cavity. For example, the tissue interface 120 may need to extend into at least one of the paracolic gutters of the abdominal cavity. In many cases, a surgeon or other health care provider may not be able to see this region of the body when deploying the tissue interface 120 and the identifier tool 402 may be used to identify where in the abdominal cavity the tissue interface is located. Using the identifier tool 402 may help to ensure that the tissue interface 120 is properly deployed within the abdominal cavity.

The handle 406 may include an indicator 408 that may be configured to actuate when the sensing portion 404 or the NFC reader 407 of the identifier tool 402 is within a sensing distance of one of the plurality of location tags 208. In other embodiments, the indicator 408 may be located at a different location on the indicator 408. When the sensing portion 404 is within the sensing distance of one of the plurality of location tags 208, the one of the plurality of location tags 208 may communicate with the identifier tool 402. For example, the one of the plurality of location tags 208 may communicate a unique identifier 410 and treatment data 412 with the identifier tool 402 when the identifier tool 402 is within the sensing distance of the one of the plurality of location tags 208.

As shown in FIG. 4, the indicator 408 may be an LED that may emit light 414 when the sensing portion 404 of the identifier tool 402 is proximate to one of the plurality of location tags 208. In other embodiments, the indicator 408 may be another type of light or another type of indicator configured to output an indication that the sensing portion 404 of the identifier tool 402 is within the sensing distance of one of the plurality of location tags 208. For example, in some embodiments, the indicator 408 may be a speaker that is configured to output a sound when the sensing portion 404 of the identifier tool 402 is proximate to one of the plurality of location tags 208. In some embodiments, the sensing distance may be about 4 inches. In other embodiments, the sensing distance may be less than or greater than about 4 inches.

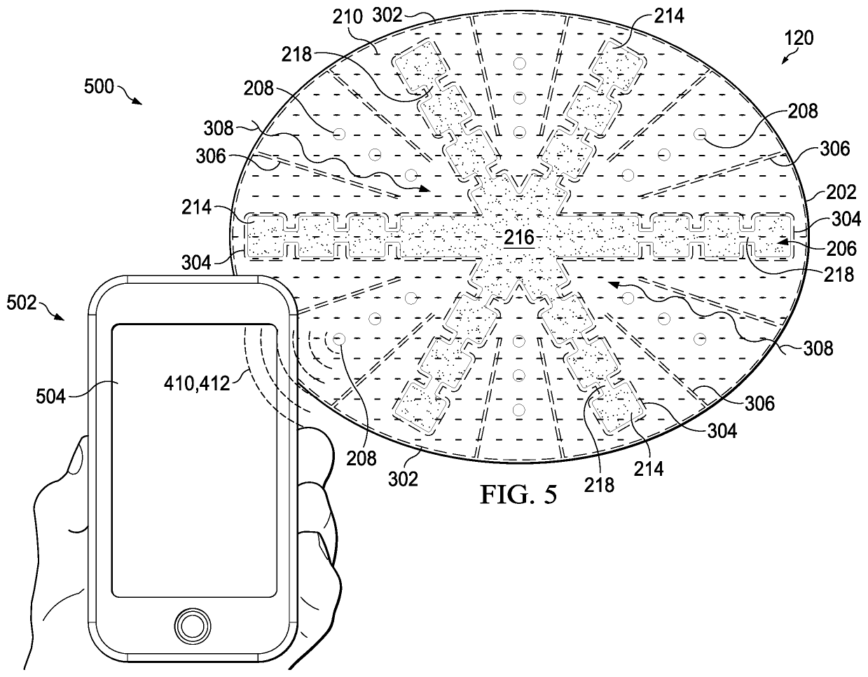

FIG. 5 is a perspective view of another embodiment of a tissue interface kit 500. The tissue interface kit 500 may include a smart identifier tool 502 and the tissue interface 120 of FIGS. 2 and 3. The smart identifier tool 502 may be configured to communicate and interact with the plurality of location tags 208 of the tissue interface 120. In some embodiments, the smart identifier tool 502 may be a smart phone that includes NFC technology that may enable the smart phone to interact and communicate with NFC devices. Similar to the identifier tool 402 described above, the smart identifier tool 502 may need to be within the sensing distance of one of the plurality of location tags 208 in order to identify the one of the plurality of location tags 208. In some embodiments, the smart identifier tool 502 may be configured to locate each location tag 208 of the plurality of location tags 208 separately from another location tag 208 of the plurality of location tags 208.

When the smart identifier tool 502 is within the sensing distance of one of the plurality of location tags 208, the one of the plurality of location tags 208 may communicate the unique identifier 410 and the treatment data 412 with the smart identifier tool 502. In some embodiments, the smart identifier tool 502 may be configured to display the unique identifier 410 of each location tag 208 of the plurality of location tags 208. For example, when the smart identifier tool 502 is within the sensing distance of one of the plurality of location tags 208, the smart identifier tool 502 may display the unique identifier 410 of the one of the plurality of location tags 208 on a user interface 504 of the smart identifier tool 502.

In some embodiments, the smart identifier tool 502 may receive the treatment data 412 from each location tag 208 of the plurality of location tags 208 when the smart identifier tool 502 is within the sensing distance of one of the plurality of location tags 208. In some embodiments, the treatment data 412 may be outputted by the smart identifier tool 502. For example, the user interface 504 of the smart identifier tool 502 may display the treatment data 412 collected from the plurality of location tags 208. In other embodiments, the smart identifier tool 502 may communicate wirelessly or via a wired connection with a computer or another device capable of receiving and displaying the treatment data 412. The treatment data 412 may then be reviewed by a user or a health care provider so that the tissue site or the abdominal cavity can be monitored without opening the tissue site up to be viewed by the user of the health care provider.

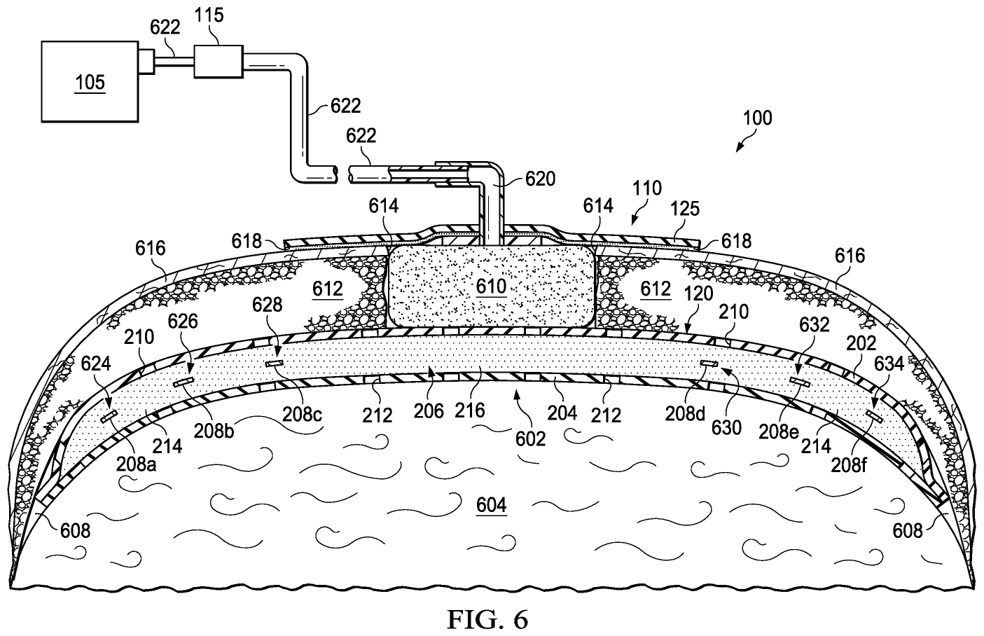

FIG. 6 is a cut away view of the therapy system of FIG. 1 deployed at a tissue site that comprises an abdominal cavity 602. The tissue interface 120 is flexible and can be inserted into the abdominal cavity 602. In some embodiments, an identifier tool such as the smart identifier tool 502, shown in FIG. 5, may be used to monitor the tissue interface 120 as it is being deployed into the abdominal cavity 602 to ensure that the tissue interface 120 is properly deployed into the abdominal cavity 602. In the example of FIG. 6, the tissue interface 120 is supported by abdominal contents 604. A portion of the tissue interface 120, such as one or more of the spacer legs 214, may be disposed in or proximate to a paracolic gutter 608.

In the example of FIG. 6, the dressing 110 includes a filler manifold 610, which can be fluidly coupled to the tissue interface 120 and can be configured to deliver negative pressure through an abdominal wall 612. For example, the filler manifold 610 may be inserted through an opening 614 in the abdominal wall 612 and disposed adjacent to the tissue interface 120 in fluid communication with at least some of the first plurality of perforations 210 of the first contact layer 202. The cover 125 may be placed over the opening 614 in the abdominal wall 612 and sealed to epidermis 616 around the opening 614. For example, an attachment device such as an adhesive layer 618 may be disposed around a perimeter of the cover 125 to secure the cover 125 to the epidermis 616.

FIG. 6 further illustrates an example of a dressing interface 620 fluidly coupling the dressing 110 to a fluid conductor 622. The dressing interface 620 may be, as one example, a port or connector, which permits the passage of fluid from the filler manifold 610 to the fluid conductor 622 and vice versa. In some embodiments, the dressing interface 620 may be an elbow connector. Fluid collected from the abdominal cavity 602 may enter the fluid conductor 622 via the dressing interface 620. In other examples, the therapy system 100 may omit the dressing interface 620, and the fluid conductor 622 may be inserted directly through the cover 125 and into the filler manifold 610. In some examples, the fluid conductor 622 may have more than one lumen. For example, the fluid conductor 622 may have one lumen for negative pressure and liquid transport and one or more lumens for communicating pressure to a pressure sensor.

A negative pressure may be applied to the central body 216 or elsewhere to cause fluid flow through the first plurality of perforations 210 and the second plurality of perforations 212. The first plurality of perforations 210 and the second plurality of perforations 212 can allow fluid to be collected or distributed through and across the first contact layer 202 and the second contact layer 204 while negative pressure is being delivered to the tissue interface 120. Fluid can move directly or indirectly towards the negative-pressure source 105 through the first plurality of perforations 210 and the second plurality of perforations 212. In some examples, additional features such as the directional bonds 306 may direct flow toward the central body 216. For example, fluid can move through the spacer layer 206, through micro-channels formed between the first contact layer 202 and the second contact layer 204, or both. Negative pressure may be distributed more directly through the spacer layer 206 and can be the dominant pathway. In some examples, the spacer layer 206 may be omitted and fluid can move through micro-channels formed between the first contact layer 202 and the second contact layer 204.

The plurality of location tags 208 may be disposed within the abdominal cavity 602 at different treatment areas within the abdominal cavity 602. For example, there may be a first treatment area 624, a second treatment area 626, a third treatment area 628, a fourth treatment area 630, a fifth treatment area 632, and a sixth treatment area 634 shown in FIG. 6. There may be a first location tag 208a disposed proximate to the first treatment area 624. The first location tag 208a may be configured to collect treatment data associated with the first treatment area 624. An identifier tool such as the smart identifier tool 502 may be configured to communicate with the first location tag 208a to collect treatment data associated with the first treatment area 624. Similarly, there may be a second location tag 208b disposed proximate to the second treatment area 626 that may be configured to collect treatment data associated with the second treatment area 626, a third location tag 208c disposed proximate to the third treatment area 628 that may be configured to collect treatment data associated with the third treatment area 628, a fourth location tag 208d disposed proximate to the fourth treatment area 630 that may be configured to collect treatment data associated with the fourth treatment area 630, a fifth location tag 208e disposed proximate to the fifth treatment area 632 that may be configured to collect treatment data associated with the fifth treatment area 632, and a sixth location tag 208f disposed proximate to the sixth treatment area 634 that may be configured to collect treatment data associated with the sixth treatment area 634. In other embodiments, there may be additional treatment areas within the abdominal cavity 602 that may contain additional location tags 208.

Also described herein is a method of placing the tissue interface 120 into the abdominal cavity 602. The method may include inserting the tissue interface 120 into the abdominal cavity 602. The tissue interface 120 can include the first contact layer 202, the second contact layer 204, the spacer layer 206, and the plurality of location tags 208. The first contact layer 202 can include the first plurality of perforations 210 and the second contact layer 204 can include the second plurality of perforations 212. The spacer layer 206 and the plurality of location tags 208 can be disposed between the first contact layer 202 and the second contact layer 204. The method can further include locating, with the smart identifier tool 502, each location tag 208 of the plurality of location tags 208 to determine an orientation of the tissue interface 120 in the abdominal cavity 602 and adjusting the orientation of the tissue interface 120 to place the tissue interface 120 in a plurality of treatment areas within the abdominal cavity 602.

In some example embodiments, the method can further include collecting treatment data associated with the abdominal cavity 602 from each location tag 208 of the plurality of location tags 208 using the smart identifier tool 502. The method can further include locating a unique identifier of each of the plurality of location tags 208 at each of the plurality of treatment areas using the smart identifier tool 502. The method can further include assigning the treatment data to each of the plurality of treatment areas using the unique identifier and outputting, with the smart identifier tool 502, the treatment data at each of the plurality of treatment areas.

The systems, apparatuses, and methods described herein may provide significant advantages. For example, the plurality of location tags 208 may allow for a health care provider to have more confidence that the tissue interface 120 is properly deployed within an abdominal cavity. Additionally, the plurality of location tags 208 may provide improved confidence about the state of healing of the abdominal cavity by collecting and communicating treatment data from the treatment areas containing the plurality of location tags 208. Further, the plurality of location tags 208 may allow for this increased confidence without requiring power at the tissue interface 120 because of the NFC technology.

While shown in a few illustrative embodiments, a person having ordinary skill in the art will recognize that the systems, apparatuses, and methods described herein are susceptible to various changes and modifications that fall within the scope of the appended claims. Moreover, descriptions of various alternatives using terms such as “or” do not require mutual exclusivity unless clearly required by the context, and the indefinite articles “a” or “an” do not limit the subject to a single instance unless clearly required by the context. Components may also be combined or eliminated in various configurations for purposes of sale, manufacture, assembly, or use. For example, in some configurations the dressing 110, the container 115, or both may be eliminated or separated from other components for manufacture or sale. In other example configurations, the controller 130 may also be manufactured, configured, assembled, or sold independently of other components.

The appended claims set forth novel and inventive aspects of the subject matter described above, but the claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described in the context of some embodiments may also be omitted, combined, or replaced by alternative features serving the same, equivalent, or similar purpose without departing from the scope of the invention defined by the appended claims.

1. A tissue interface for use in an abdominal cavity, the tissue interface comprising:
a first contact layer comprising a first plurality of perforations;
a second contact layer comprising a second plurality of perforations;
a spacer layer disposed between the first contact layer and the second contact layer; and
a plurality of location tags configured to be located by an identifier tool and to be positioned in a plurality of treatment areas within the abdominal cavity.

2. The tissue interface of claim 1, wherein the spacer layer comprises a central portion and a plurality of appendages radiating from the central portion and configured to extend to a plurality of zones within the abdominal cavity.

3. The tissue interface of claim 2, wherein the plurality of location tags are disposed between the plurality of appendages of the spacer layer.

4. The tissue interface of claim 1, wherein each location tag of the plurality of location tags is a near field communication (NFC) device.

5. The tissue interface of claim 1, wherein each location tag of the plurality of location tags is encoded with a unique identifier.

6. The tissue interface of claim 1, wherein the plurality of location tags are disposed between the first contact layer and the second contact layer and isolated from the abdominal cavity by the first contact layer and the second contact layer.

7. The tissue interface of claim 1, wherein the identifier tool is configured to locate each location tag of the plurality of location tags separately from another location tag of the plurality of location tags.

8. The tissue interface of claim 1, wherein the identifier tool is configured to locate one of the location tags of the plurality of location tags when the identifier tool is within a sensing distance thereof.

9. The tissue interface of claim 1, wherein the plurality of location tags are further configured to collect treatment data associated with the plurality of treatment areas within the abdominal cavity.

10. The tissue interface of claim 9, wherein the treatment data comprises at least one of a pressure in the abdominal cavity, a temperature in the abdominal cavity, a pH in the abdominal cavity, or a presence of fluid in the abdominal cavity.

11. The tissue interface of claim 9, wherein the plurality of location tags are configured to communicate the treatment data with the identifier tool.

12. A tissue interface kit comprising:
a tissue interface, comprising:
a first contact layer comprising a first plurality of perforations;
a second contact layer comprising a second plurality of perforations;
a spacer layer disposed between the first contact layer and the second contact layer; and
a plurality of location tags disposed between the first contact layer and the second contact layer and configured to be positioned in a plurality of treatment areas within an abdominal cavity; and
an identifier tool configured to sense a location of one of the plurality of location tags of the tissue interface at each of the treatment areas.

13. The tissue interface kit of claim 12, wherein the identifier tool comprises a handheld device comprising a locator and an indicator, wherein the indicator is configured to actuate when the locator is within a sensing distance of one of the location tags of the plurality of location tags.

14. The tissue interface kit of claim 12, wherein the identifier tool comprises a smart phone configured to communicate with each location tag of the plurality of location tags.

15. The tissue interface kit of claim 14, wherein the plurality of location tags are further configured to collect treatment data from each of the plurality of treatment areas within the abdominal cavity.

16. The tissue interface kit of claim 15, wherein the identifier tool is configured to display the treatment data of one of the location tags of the plurality of location tags when the identifier tool is within a sensing distance thereof.

17. The tissue interface kit of claim 12, wherein each location tag of the plurality of location tags is encoded with a unique identifier.

18. The tissue interface kit of claim 17, wherein the identifier tool is configured to display the unique identifier of one of the location tags of the plurality of location tags when the identifier tool is within a sensing distance thereof.

19. A method of placing a tissue interface into an abdominal cavity comprising:
inserting the tissue interface into the abdominal cavity, the tissue interface comprising:
a first contact layer comprising a first plurality of perforations;
a second contact layer comprising a second plurality of perforations;
a spacer layer disposed between the first contact layer and the second contact layer; and
a plurality of location tags disposed between the first contact layer and the second contact layer;
locating, with an identifier tool, each location tag of the plurality of location tags to determine an orientation of the tissue interface in the abdominal cavity; and
adjusting the orientation of the tissue interface to place the tissue interface in a plurality of treatment areas within the abdominal cavity.

20. The method of claim 19, further comprising:
collecting treatment data associated with the abdominal cavity from each location tag of the plurality of location tags using the identifier tool;
locating a unique identifier of each of the plurality of location tags at each of the plurality of treatment areas using the identifier tool;
assigning the treatment data to each of the plurality of treatment areas using the unique identifier; and
outputting, with the identifier tool, the treatment data at each of the plurality of treatment areas.

21. (canceled)
